(19) United States
(12) Patent Application Publication
(10) Pub. No.: US 2022/0387244 A1
(43) Pub. Date: Dec. 8, 2022

(54) WALKING TRAINING SYSTEM, CONTROL METHOD THEREOF, AND CONTROL PROGRAM

(71) Applicant: TOYOTA JIDOSHA KABUSHIKI KAISHA, Toyota-shi (JP)

(72) Inventor: Taiga MATSUMOTO, Nagoya-shi (JP)

(73) Assignee: TOYOTA JIDOSHA KABUSHIKI KAISHA, Toyota-shi (JP)

(21) Appl. No.: 17/713,929

(22) Filed: Apr. 5, 2022

(30) Foreign Application Priority Data
Jun. 3, 2021 (JP) 2021-093492

(51) Int. Cl. A61H 1/02; A63B 22/02; A63B 24/00; A61B 5/11; B25J 9/00

(52) U.S. Cl. CPC A61H 1/0262; A63B 22/02; A63B 24/0062; A61B 5/112; B25J 9/0006; A63B 2220/50; A61H 2201/1642; A61B 2505/09; A63B 2214/00

(57) ABSTRACT

A walking training device according to the present embodiment includes: a robot leg attached to one leg of a trainee; a treadmill; a load distribution sensor that detects a distribution of a load received from a sole of the trainee riding on the belt of the treadmill; and a walking state determination unit that determines whether the one leg has switched from a standing state to a swinging state based on a state of increase in a load detected by the load distribution sensor and received from another leg of the trainee performing walking training; and a control unit that starts bending control for the swinging state of the robot leg when the walking state determination unit determines that the one leg has switched from the standing state to the swinging state.

### CROSS-REFERENCE TO RELATED APPLICATION

This application claims priority to Japanese Patent Application No. 2021-093492 filed on Jun. 3, 2021, incorporated herein by reference in its entirety.

### BACKGROUND

### 1. Technical Field

The present disclosure relates to a walking training system, a control method thereof, and a control program.

### 2. Description of Related Art

Japanese Unexamined Patent Application Publication No. 2016-73525 (JP 2016-73525 A) discloses a walking training system including a dynamic balance ability evaluation device for evaluating the dynamic balance ability of a user based on the change over time of a predetermined body part due to walking of the user, and a treadmill for the user to perform walking training. This walking training system includes a pressure sensor on the belt of the treadmill, and detects the force with which the user kicks the belt (floor reaction force) from the measured value of the pressure sensor, for example.

### SUMMARY

The response performance of the pressure sensor when the pressure sensor is unloaded is usually lower than that when the pressure sensor is loaded. Therefore, in the related art, even when one leg of the user performing the walking training shifts from the standing state to the swinging state, the load received from the one leg is not removed and is unintentionally detected. Thus, the timing of switching of the one leg from the standing state to the swinging state cannot be accurately detected. That is, with the related art, the walking state of the user (trainee) cannot be accurately determined. As a result, there is a problem that the related art cannot provide effective walking training to the user.

The present disclosure has been made in view of the above background, and an object of the present disclosure is to provide a walking training system capable of providing effective training to a trainee by improving accuracy of determining the walking state of the trainee, a control method thereof, and a control program.

A walking training system according to an embodiment of the present disclosure includes: a robot leg attached to one leg of a trainee; a treadmill; a load distribution sensor that is attached to the treadmill and detects a distribution of a load received from a sole of the trainee riding on a belt of the treadmill; a walking state determination unit that determines whether the one leg has switched from a standing state to a swinging state based on a state of increase in a load detected by the load distribution sensor and received from another leg of the trainee performing walking training; and a control unit that starts bending control for the swinging state of the one leg by the robot leg when the walking state determination unit determines that the one leg has switched from the standing state to the swinging state. This walking training system can accurately detect the timing of switching of the leg of the trainee performing walking training from the standing state to the swinging state. Therefore, it is possible to improve the accuracy of determining the walking state of the trainee, and as a result, it is possible to provide effective walking training to the trainee. For example, this walking training system can accurately detect the timing of switching of the leg to which the robot leg is attached from the standing state to the swinging state. Therefore, the robot leg can be bent and extended at an appropriate timing, and as a result, it is possible to provide effective walking training to the trainee.

The walking state determination unit determines that the one leg has switched from the standing state to the swinging state when the load received from the other leg becomes equal to or more than a predetermined load.

The walking state determination unit determines that the one leg has switched from the standing state to the swinging state when the load received from the other leg becomes equal to or more than a predetermined ratio of a maximum value of a load received from the one leg.

The walking state determination unit determines that the one leg has switched from the standing state to the swinging state when a center-of-gravity position of the load detected by the load distribution sensor comes in a predetermined region including a position of the other leg.

A method for controlling a walking training system according to an embodiment of the present disclosure includes: a step of using a load distribution sensor attached to a treadmill to detect a distribution of a load received from a sole of a trainee riding on a belt of the treadmill; a step of determining whether one leg to which a robot leg is attached has switched from a standing state to a swinging state based on a state of increase in a load received from another leg that is different from the one leg; and a step of starting bending control for the swinging state of the one leg by the robot leg when determining that the one leg has switched from the standing state to the swinging state. The method for controlling a walking training system can accurately detect the timing of switching of the leg of the trainee performing walking training from the standing state to the swinging state. Therefore, it is possible to improve the accuracy of determining the walking state of the trainee, and as a result, it is possible to provide effective walking training to the trainee. For example, the method for controlling a walking training system can accurately detect the timing of switching of the leg to which the robot leg is attached from the standing state to the swinging state. Therefore, the robot leg can be bent and extended at an appropriate timing, and as a result, it is possible to provide effective walking training to the trainee.

A control program according to an embodiment of the present disclosure causes a computer to execute: a process of using a load distribution sensor attached to a treadmill to detect a distribution of a load received from a sole of a trainee riding on a belt of the treadmill; a process of determining whether one leg to which a robot leg is attached has switched from a standing state to a swinging state based on a state of increase in a load received from another leg that is different from the one leg; and a process of starting bending control for the swinging state of the one leg by the robot leg when determining that the one leg has switched from the standing state to the swinging state. The control program can accurately detect the timing of switching of the leg of the trainee performing walking training from the standing state to the swinging state. Therefore, it is possible to improve the accuracy of determining the walking state of the trainee, and as a result, it is possible to provide effective walking training to the trainee. For example, the control program can accurately detect the timing of switching of the leg to which the robot leg is attached from the standing state to the swinging state. Therefore, the robot leg can be bent and extended at an appropriate timing, and as a result, it is possible to provide effective walking training to the trainee.

According to the present disclosure, it is possible to provide a walking training system capable of providing effective training to a trainee by improving accuracy of determining the walking state of the trainee, a control method thereof, and a control program.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is an overall conceptual diagram showing a configuration example of a walking training device according to a first embodiment;

FIG. 2 is a schematic side view of a part of a treadmill provided in the walking training device shown in FIG. 1;

FIG. 3 is a schematic perspective view showing a configuration example of a walking assist device provided in the walking training device shown in FIG. 1;

FIG. 4 is a block diagram showing a system configuration example of the walking training device shown in FIG. 1;

FIG. 5 is a timing chart illustrating an influence of low response performance when a load distribution sensor is unloaded;

FIG. 6 is a timing chart obtained by enlarging a part of FIG. 5;

FIG. 7 is a timing chart showing an example of a method of determining a walking state of a trainee by the walking training device shown in FIG. 1;

FIG. 8 is a timing chart obtained by enlarging a part of FIG. 7;

FIG. 9 is a schematic plan view illustrating another example of the method of determining the walking state of the trainee by the walking training device shown in FIG. 1; and

FIG. 10 is a timing chart showing the other example of the method of determining the walking state of the trainee by the walking training device shown in FIG. 1.

### DETAILED DESCRIPTION OF EMBODIMENTS

Hereinafter, the present disclosure will be described through embodiments of the disclosure, but the disclosure according to the scope of the claims is not limited to the following embodiments. Moreover, not all of the configurations described in the embodiments are indispensable as means for solving the problem. For the sake of clarity, omission and simplification are made as appropriate in the following description and drawings. In the drawings, the same elements are designated by the same reference signs, and duplicate descriptions are omitted as necessary.

### First Embodiment

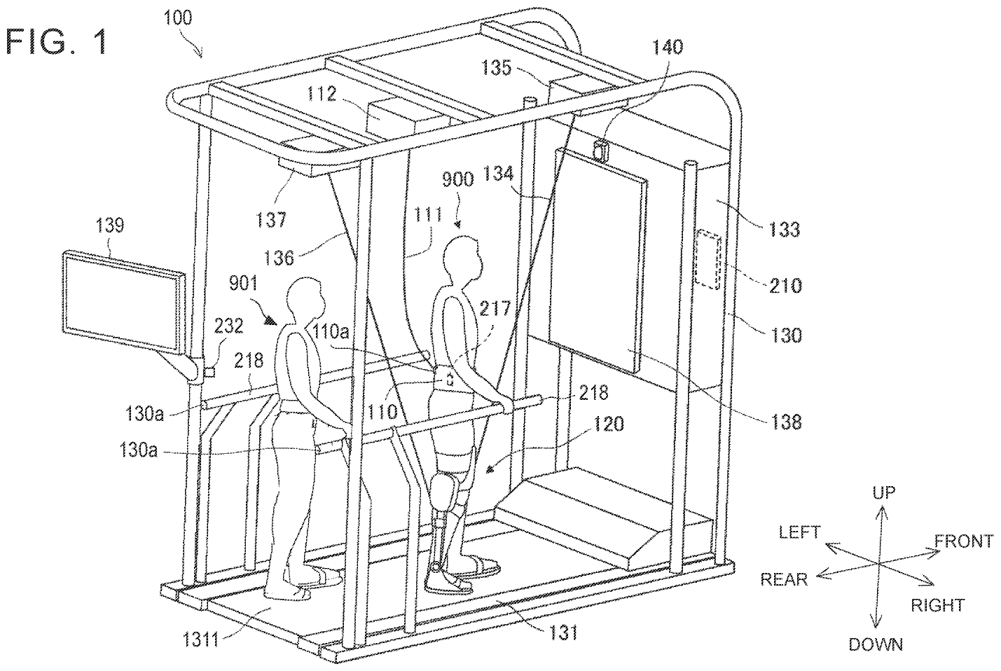

FIG. 1 is an overall conceptual diagram showing a configuration example of a walking training device according to a first embodiment. A walking training device 100 according to the present embodiment is a specific example of a rehabilitation support device that supports the rehab (rehabilitation) of a trainee (user) 900, and is particularly a specific example of a walking training device that supports walking training. The walking training device 100 is a device for the trainee 900 who is a hemiplegic patient suffering from paralysis in one leg to perform walking training in accordance with the guidance of a training staff 901. Here, the training staff 901 can be, for example, a therapist (physiotherapist) or a doctor, and assists the training of the trainee by guidance or caregiving. Therefore, the training staff 901 may be called a training instructor, a training caregiver, or a training assistant. The walking training device 100 can also be called a walking training system. The up-down direction, the right-left direction and the front-rear direction in the following description are directions based on the direction of the trainee 900.

The walking training device 100 mainly includes a control panel 133 attached to a frame 130 constituting the entire skeleton, a treadmill 131 on which the trainee 900 walks, and a walking assist device (robot leg) 120 that is attached to an affected leg that is a leg of the trainee 900 on the paralyzed side.

The treadmill 131 is a device that prompts the trainee 900 to walk, and the trainee 900 who performs walking training rides on a belt 1311 and attempts a walking motion in accordance with the movement of the belt 1311. The training staff 901 can stand on the belt 1311 behind the trainee 900 and perform a walking motion together with the trainee 900 as shown in FIG. 1, for example. However, it is usually preferable that the training staff 901 be in a state in which it is easy to perform caregiving to the trainee 900, that is, standing over the belt 1311.

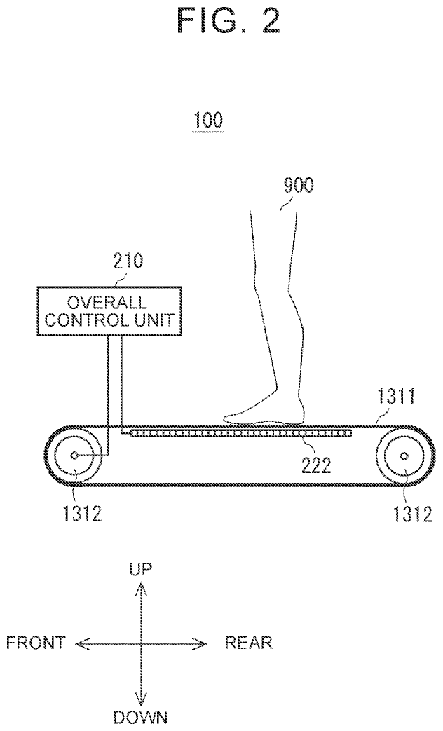

FIG. 2 is a schematic side view of a part of the treadmill 131. As shown in FIG. 2, the treadmill 131 includes at least the ring-shaped belt 1311, a pulley 1312, and a motor (not shown). Further, a load distribution sensor 222 is installed on the inner side of the belt 1311 (on the lower side of the belt 1311 on the surface of which the trainee 900 rides) so as not to move together with the belt 1311. However, the load distribution sensor 222 may be provided on the upper side of the belt 1311 so as to move together with the belt 1311.

The load distribution sensor 222 is composed of a plurality of sensors, and these sensors are arranged in a matrix on the lower side of the belt 1311 that supports the sole of the trainee 900. By using these sensors, the load distribution sensor 222 can detect the magnitude and the distribution of the surface pressure (load) received from the sole of the trainee 900. For example, the load distribution sensor 222 is a resistance change detection-type load detection sheet in which a plurality of electrodes is arranged in a matrix. From the detection result of the load distribution sensor 222, it is possible to determine the walking state of the trainee 900 (whether each leg is in a standing state or a swinging state, and the like). The details of the method of determining the walking state of the trainee 900 based on the detection result of the load distribution sensor 222 will be described later.

In the treadmill 131, for example, an overall control unit 210, which will be described later, determines the walking state of the trainee 900 based on the detection result of the load distribution sensor 222, and uses a motor (not shown) to rotate the pulley 1312 in accordance with the walking state, thereby rotating (moving) the ring-shaped belt 1311. As a result, the trainee 900 can perform walking training without stepping out from the belt 1311.

The frame 130 stands on the treadmill 131 installed on the floor surface, supports the control panel 133 housing the overall control unit 210 that controls the motor and the sensor, and supports a training monitor 138 that is, for example, a liquid crystal panel that presents the training progress and the like to the trainee 900. Further, the frame 130 supports a front tension unit 135 at the front of the overhead portion of the trainee 900, a harness tension unit 112 at the overhead portion, and a rear tension unit 137 at the rear of the overhead portion. The frame 130 also includes handrails 130a for the trainee 900 to grab.

The handrails 130a are arranged on right and left sides of the trainee 900. Each handrail 130a is disposed to extend in a direction parallel to the walking direction of the trainee 900. The position of the handrail 130a in the up-down direction and the right-left direction can be adjusted. That is, the handrails 130a can include a mechanism for changing their height and width. Further, the handrail 130a can be configured such that the height of the handrail 130a is adjusted to make the height of the front side and the height of the rear side in the walking direction different so as to change the inclination angle thereof, for example. For example, the handrail 130a can be provided with an inclination angle that gradually increases along the walking direction.

Further, the handrail 130a is provided with a handrail sensor 218 for detecting the load received from the trainee 900. For example, the handrail sensor 218 can be a resistance change detection-type load detection sheet in which electrodes are arranged in a matrix. Further, the handrail sensor 218 can be a six-axis sensor in which a three-axis acceleration sensor (x, y, z) and a three-axis gyro sensor (roll, pitch, yaw) are combined. However, the type and the installation position of the handrail sensor 218 are not limited.

A camera 140 functions as an imaging unit for observing the whole body of the trainee 900. The camera 140 is installed near the training monitor 138 so as to face the trainee. The camera 140 captures still images and moving images of the trainee 900 during training. The camera 140 includes a set of a lens and an imaging element that provides such an angle of view that the whole body of the trainee 900 can be captured. The imaging element is, for example, a complementary metal-oxide-semiconductor (CMOS) image sensor that converts an optical image on an image plane into an image signal.

With the coordinated operation of the front tension unit 135 and the rear tension unit 137, the load of the walking assist device 120 is offset so as not to be a burden on the affected leg, and further, the forward swing motion of the affected leg is assisted in accordance with the degree of the setting.

One end of a front wire 134 is connected to a winding mechanism of the front tension unit 135, and the other end is connected to the walking assist device 120. The winding mechanism of the front tension unit 135 winds and unwinds the front wire 134 in accordance with the movement of the affected leg by turning on and off a motor (not shown). Similarly, one end of a rear wire 136 is connected to a winding mechanism of the rear tension unit 137, and the other end is connected to the walking assist device 120. The winding mechanism of the rear tension unit 137 winds and unwinds the rear wire 136 in accordance with the movement of the affected leg by turning on and off a motor (not shown). With such a coordinated operation of the front tension unit 135 and the rear tension unit 137, the load of the walking assist device 120 is offset so as not to be a burden on the affected leg, and further, the forward swing motion of the affected leg is assisted in accordance with the degree of the setting.

For example, as an operator, the training staff 901 sets the level of assistance to high, for a trainee who has severe paralysis. When the assist level is set to high, the front tension unit 135 winds up the front wire 134 with a relatively large force in accordance with the forward swing timing of the affected leg. As the training progresses and assistance becomes no longer needed, the training staff 901 sets the assist level to the minimum. When the assist level is set to the minimum, the front tension unit 135 winds up the front wire 134 with a force to cancel the weight of the walking assist device 120 in accordance with the forward swing timing of the affected leg.

The walking training device 100 further includes a fall prevention harness device composed of a brace 110, a harness wire 111, and a harness tension unit 112.

The brace 110 is a belt wrapped around the abdomen of the trainee 900 and is fixed to the waist portion by, for example, a hook-and-loop fastener. The brace 110 includes a connecting hook 110a for connecting one end of the harness wire 111 that is a hanger, and can also be referred to as a hanger belt. The trainee 900 wears the brace 110 such that the connecting hook 110a is located on the rear back portion.

One end of the harness wire 111 is connected to the connecting hook 110a of the brace 110, and the other end is connected to the winding mechanism of the harness tension unit 112. The winding mechanism of the harness tension unit 112 winds and unwinds the harness wire 111 by turning on and off a motor (not shown). With such a configuration, when the trainee 900 is about to fall, the fall prevention harness device winds up the harness wire 111 in accordance with the instruction of the overall control unit 210 that detects the movement, supports the upper body of the trainee 900 with the brace 110, and suppresses the trainee 900 from falling.

The brace 110 includes a posture sensor 217 for detecting the posture of the trainee 900. The posture sensor 217 is, for example, a combination of a gyro sensor and an acceleration sensor, and outputs an inclination angle of the abdomen on which the brace 110 is attached with respect to the direction of gravity.

The management monitor 139 is a display input device mainly for monitoring and operation by the training staff 901, and is attached to the frame 130. The management monitor 139 is, for example, a liquid crystal panel, and a touch panel is provided on the surface thereof. The management monitor 139 displays various menu items related to training settings, various parameter values at the time of training, training results, and the like. Further, an emergency stop button 232 is provided near the management monitor 139. When the training staff 901 presses the emergency stop button 232, an emergency stop of the walking training device 100 is performed.

The walking assist device 120 is attached to the affected leg of the trainee 900 and assists the trainee 900 in walking by reducing the load of extension and bending at the knee joint of the affected leg. The walking assist device 120 transmits data on the leg movement acquired through walking training to the overall control unit 210, or drives the joint portion in accordance with the instruction from the overall control unit 210. The walking assist device 120 can also be connected to a hip joint (a connecting member including a rotating portion) attached to the brace 110 that is a part of the fall prevention harness device via a wire or the like.

### Details of Walking Assist Device 120

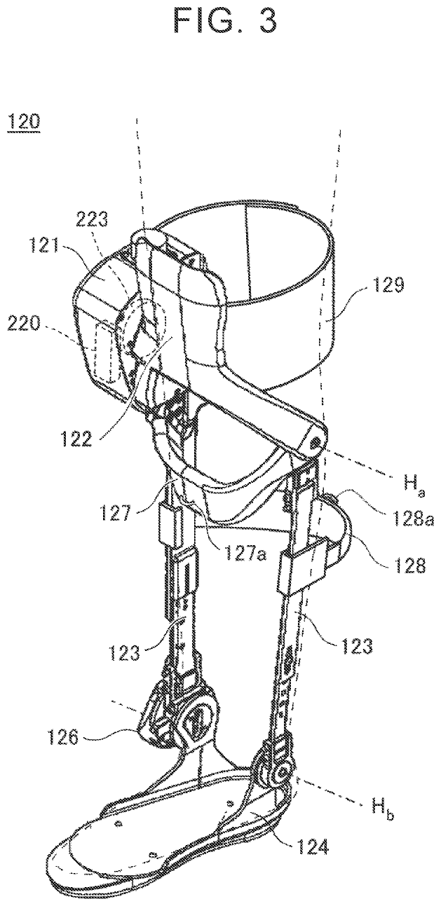

FIG. 3 is a schematic perspective view showing a configuration example of the walking assist device 120. The walking assist device 120 mainly includes a control unit 121 and a plurality of frames that supports various parts of the affected leg. The walking assist device 120 is also referred to as a robot leg.

The control unit 121 includes an auxiliary control unit 220 that controls the walking assist device 120, and also includes a motor (not shown) that generates a driving force for assisting the extension motion and the bending motion of the knee joint. The frames that support various parts of the affected leg include an upper leg frame 122 and lower leg frames 123 that are pivotably connected to the upper leg frame 122. The frames further include a foot flat frame 124 pivotably connected to the lower leg frames 123, a front connecting frame 127 for connecting the front wire 134, and a rear connecting frame 128 for connecting the rear wire 136.

The upper leg frame 122 and the lower leg frames 123 pivot relative to each other around a hinge axis Hₐ shown in the figure. The motor of the control unit 121 rotates following the instruction of the auxiliary control unit 220 to force the upper leg frame 122 and the lower leg frames 123 to relatively open or close around the hinge axis Hₐ. The angle sensor 223 housed in the control unit 121 is, for example, a rotary encoder, and detects the angle between the upper leg frame 122 and the lower leg frames 123 around the hinge axis Hₐ. The lower leg frames 123 and the foot flat frame 124 pivot relative to each other around the hinge axis H_{b} shown in the figure. The relative pivot angle range is adjusted in advance by an adjusting mechanism 126.

The front connecting frame 127 is provided so as to extend in the right-left direction on the front side of the upper leg and connect to the upper leg frame 122 at both ends. The front connecting frame 127 is further provided with a connecting hook 127a for connecting the front wire 134, around the center in the right-left direction. The rear connecting frame 128 is provided so as to extend in the right-left direction on the rear side of the lower leg and connect to the lower leg frames 123 extending in the up-down direction at both ends. The rear connecting frame 128 is further provided with a connecting hook 128a for connecting the rear wire 136, around the center in the right-left direction.

The upper leg frame 122 includes an upper leg belt 129. The upper leg belt 129 is a belt integrally provided on the upper leg frame, and is wrapped around the upper leg portion of the affected leg to fix the upper leg frame 122 to the upper leg portion. This suppresses the entire walking assist device 120 from shifting with respect to the leg of the trainee 900.

### System Configuration Example of Walking Training Device 100

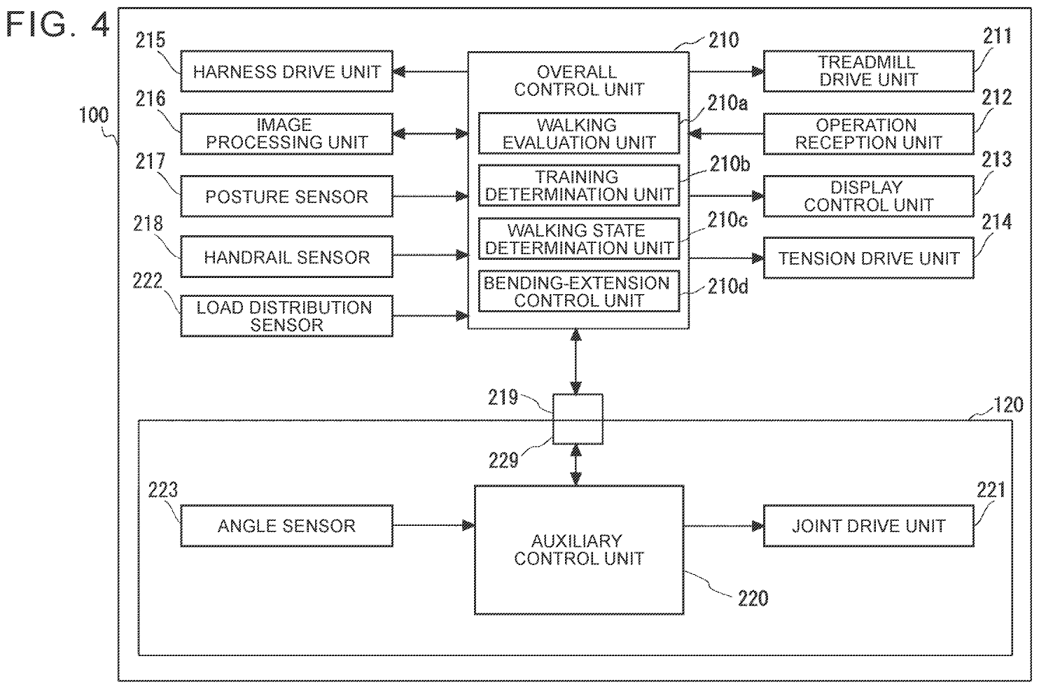

Subsequently, a system configuration example of the walking training device 100 will be described with reference to FIG. 4. FIG. 4 is a block diagram showing the system configuration example of the walking training device 100.

As shown in FIG. 4, the system configuration of the walking training device 100 includes the overall control unit 210, a treadmill drive unit 211, an operation reception unit 212, a display control unit 213, a tension drive unit 214, a harness drive unit 215, an image processing unit 216, the posture sensor 217, the handrail sensor 218, the load distribution sensor 222, a communication connection interface (IF) 219, and the walking assist device 120.

The overall control unit 210 is, for example, a micro processing unit (MPU), and executes control of the entire device by executing a control program read from a system memory.

The treadmill drive unit 211 includes a motor for rotating the belt 1311 of the treadmill 131 and a drive circuit thereof. The overall control unit 210 executes rotation control of the belt 1311 by transmitting a drive signal to the treadmill drive unit 211. The overall control unit 210 adjusts the rotation speed of the belt 1311 in accordance with, for example, the walking speed set by the training staff 901. Alternatively, the overall control unit 210 adjusts the rotation speed of the belt 1311 in accordance with the walking state of the trainee 900 determined based on the detection result of the load distribution sensor 222.

The operation reception unit 212 receives an input operation by the training staff 901 via an operation button provided on the device, a touch panel superimposed on the management monitor 139, an attached remote controller, or the like. The operation signal received by the operation reception unit 212 is transmitted to the overall control unit 210. The overall control unit 210 can give the instruction to switch on and off the power supply or give the instruction to start training based on the operation signal received by the operation reception unit 212. In addition, it is possible to input numerical values related to settings and select menu items. The operation reception unit 212 is not limited to the case where the input operation of the training staff 901 is received, and of course, the operation reception unit 212 can also receive the input operation of the trainee 900.

The display control unit 213 receives a display signal from the overall control unit 210, generates a display image, and displays the image on the training monitor 138 or the management monitor 139. The display control unit 213 generates an image showing the progress of training and a real-time image captured by the camera 140 in accordance with the display signal.

The tension drive unit 214 includes a motor for pulling the front wire 134 and a drive circuit thereof that are provided in the front tension unit 135, and a motor for pulling the rear wire 136 and a drive circuit thereof that are provided in the rear tension unit 137. The overall control unit 210 controls the winding of the front wire 134 and the winding of the rear wire 136 by transmitting a drive signal to the tension drive unit 214. Further, the overall control unit 210 controls the tensile force of each wire by controlling the driving torque of the motor, not limited to the winding operation. Further, the overall control unit 210 identifies the timing at which the affected leg switches from the standing state to the swinging state based on the detection result of the load distribution sensor 222, and increases or decreases the tensile force of each wire in synchronization with that timing, thereby assisting the forward swing motion of the affected leg.

The harness drive unit 215 includes a motor for pulling the harness wire 111 and a drive circuit thereof that are provided in the harness tension unit 112. The overall control unit 210 controls the winding of the harness wire 111 and the tensile force of the harness wire 111 by transmitting a drive signal to the harness drive unit 215. For example, when the trainee 900 is predicted to fall, the overall control unit 210 winds up the harness wire 111 by a certain amount to suppress the trainee from falling.

The image processing unit 216 is connected to the camera 140 and can receive an image signal from the camera 140. The image processing unit 216 receives an image signal from the camera 140 and performs image processing on the received image signal to generate image data, in accordance with the instruction from the overall control unit 210. Further, the image processing unit 216 can also perform image processing on the image signal received from the camera 140 to execute a specific image analysis, in accordance with the instruction from the overall control unit 210. For example, the image processing unit 216 detects the position of the foot (standing position) of the affected leg that is in contact with the treadmill 131 by image analysis. Specifically, for example, the standing position is calculated by extracting an image region near the tip of the foot flat frame 124 and analyzing an identification marker drawn on the belt 1311 that overlaps the tip portion.

As described above, the posture sensor 217 detects the inclination angle of the abdomen of the trainee 900 with respect to the direction of gravity, and transmits the detection signal to the overall control unit 210. The overall control unit 210 calculates the posture of the trainee 900, specifically the inclination angle of the trunk, using the detection signal from the posture sensor 217. The overall control unit 210 and the posture sensor 217 may be connected by wire or by short-range wireless communication.

The handrail sensor 218 detects a load applied to the handrail 130a. That is, a load corresponding to a portion of the weight of the trainee 900 that the trainee 900 cannot support with both legs is applied to the handrail 130a. The handrail sensor 218 detects this load and transmits a detection signal to the overall control unit 210.

As described above, the load distribution sensor 222 detects the magnitude and the distribution of the surface pressure (load) received from the sole of the trainee 900 and transmits the detection signal to the overall control unit 210. The overall control unit 210 receives and analyzes the detection signal to determine the walking state and estimate switching.

The overall control unit 210 also plays a role as a function execution unit that executes various calculations related to the control and performs the control. The overall control unit 210 includes, for example, a walking evaluation unit 210a, a training determination unit 210b, a walking state determination unit 210c, and a bending-extension control unit 210d. The walking state determination unit 210c and the bending-extension control unit 210d will be described later.

The walking evaluation unit 210a evaluates whether the walking motion of the trainee 900 is abnormal walking using the data acquired from various sensors. The training determination unit 210b determines the training result for a series of walking trainings based on, for example, the cumulative number of abnormal walking evaluated by the walking evaluation unit 210a.

The method of determining the training result and the criteria for determining the training result may be set as appropriate. For example, the training result may be determined by comparing the amount of movement of the paralyzed body portion with the reference for each walking phase. The walking phase is obtained by dividing one walking cycle for the affected leg (or a healthy leg) into a standing phase in which the leg is in the standing state, a transition phase from the standing phase to a swinging phase in which the leg is in the swinging state, a swinging phase, a transition phase from the swinging phase to the standing phase, and so on. The walking phase can be classified (determined) based on, for example, the detection result by the load distribution sensor 222. As described above, for the walking cycle, one cycle can be regarded as including the standing phase, the transition phase, the swinging phase, and the transition phase. However, it does not matter which phase is defined as the start phase. In addition, for the walking cycle, one cycle can be regarded as including, for example, a both leg-supported state, a single leg—(affected leg-)supported state, the both leg-supported state, and a single leg—(healthy leg-)supported state, and in this case, it does not matter which state is defined as the starting state.

In addition, the walking cycle focusing on the right leg or the left leg (healthy leg or affected leg) can be further divided, and can be represented by dividing the standing phase into an initial ground contact and four phases and dividing the swinging phase into three phases. The initial ground contact refers to a moment when an observed foot contacts the floor, and the four phases of the standing phase refer to a load response phase, a standing middle phase, a standing end phase, and a pre-swinging phase. The load response phase is the phase from the initial ground contact to the moment when the foot on the opposite side leaves the floor (contralateral takeoff). The standing middle phase is the phase from the contralateral takeoff to the moment when the heel of the observed foot leaves the floor (heel takeoff). The standing end phase is the phase from the heel takeoff to the initial ground contact on the opposite side. The pre-swinging phase is the phase from the initial ground contact on the opposite side to the time when the observed foot leaves the floor (takeoff). The three phases of the swinging phase refer to a swinging initial phase, a swinging middle phase, and a swinging end phase. The swinging initial phase is the phase from the end of the pre-swinging phase (the above-mentioned takeoff) to the time when both feet cross (feet crossing). The swinging middle phase is the phase from the time when the feet cross to the time when the shinbone becomes vertical (vertical shinbone). The swinging end phase is the phase from the time when the shinbone is vertical to the next initial ground contact.

The communication connection IF 219 is an interface connected to the overall control unit 210, and is an interface for providing a command to the walking assist device 120 attached to the affected leg of the trainee 900 and receiving sensor information.

The walking assist device 120 can include a communication connection IF 229 that is connected to the communication connection IF 219 by a wire or wirelessly. The communication connection IF 229 is connected to the auxiliary control unit 220 of the walking assist device 120. The communication connection IF 219 and the communication connection IF 229 are communication interfaces such as a wired local area network (LAN) or a wireless LAN conforming to the communication standards.

Further, the walking assist device 120 can include the auxiliary control unit 220, a joint drive unit 221, and the angle sensor 223. The auxiliary control unit 220 is, for example, an MPU, and controls the walking assist device 120 by executing the control program provided by the overall control unit 210. Further, the auxiliary control unit 220 notifies the overall control unit 210 of the state of the walking assist device 120 via the communication connection IF 219 and the communication connection IF 229. Further, the auxiliary control unit 220 receives a command from the overall control unit 210 and executes control of starting, stopping, and the like of the walking assist device 120.

The joint drive unit 221 includes a motor of the control unit 121 and a drive circuit thereof. The auxiliary control unit 220 transmits the drive signal to the joint drive unit 221 to force the upper leg frame 122 and the lower leg frames 123 to relatively open or close around the hinge axis Hₐ. Such motions assist the knee extension and bending motions and suppress knee collapse.

As described above, the angle sensor 223 detects the angle between the upper leg frame 122 and the lower leg frames 123 around the hinge axis Hₐ, and transmits the detection signal to the auxiliary control unit 220. The auxiliary control unit 220 receives this detection signal and calculates the opening angle of the knee joint.

The response performance of the load distribution sensor 222 when the load distribution sensor 222 is unloaded is usually lower than that when the load distribution sensor 222 is loaded. Therefore, even when one leg of the trainee 900 performing walking training leaves the belt 1311 of the treadmill 131 at the timing of switching from the standing state to the swinging state, the load distribution received from the one leg may not be removed and may be unintentionally detected. In that case, the one leg is determined as being in the standing state even though the leg has been shifted from the standing state to the swinging state. If the one leg is an affected leg to which the walking assist device (robot leg) 120 is attached, bending control for the swinging state by the walking assist device 120 cannot be started at an appropriate timing. Therefore, the trainee 900 is not be able to perform effective walking training.

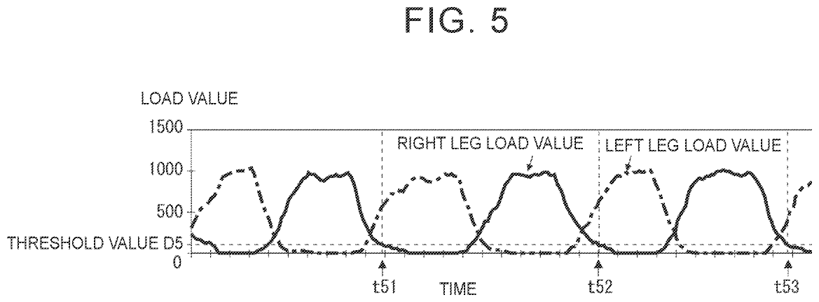

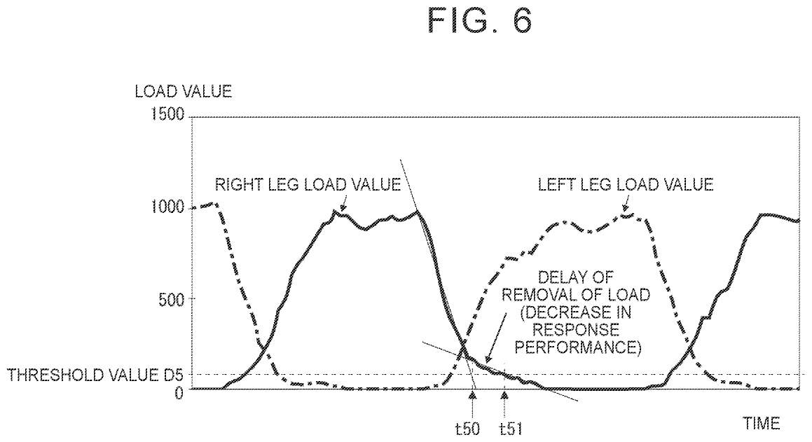

FIG. 5 is a timing chart illustrating an influence of low response performance when the load distribution sensor 222 is unloaded. FIG. 6 is a timing chart obtained by enlarging a part of FIG. 5. In the example of FIGS. 5 and 6, a case where the right leg is the affected leg to which the walking assist device 120 is attached and the left leg is a healthy leg will be described.

In the example of FIG. 5, it is determined that the right leg has switched from the standing state to the swinging state at the timing when the load value of the right leg, which is the affected leg, decreases to a value less than a threshold value D5 (time t51, t52, t53). However, as can be seen from the timing chart of FIG. 6 obtained by enlarging the vicinity of time t51, the right leg has actually shifted to the swinging state at time t50 that is before time t51. Despite that, it is determined that the right leg has shifted to the swinging state at time t51 due to the delay of removal of load. This makes the start timing of the bending control for the swinging state by the walking assist device 120 attached to the right leg delay, so that the trainee 900 cannot perform effective walking training.

Thus, in the present embodiment, the walking state determination unit 210c determines whether the leg (affected leg) to which the walking assist device 120 is attached has switched from the standing state to the swinging state, based on the state of increase in the load received by the load distribution sensor 222 from the leg (healthy leg) to which the walking assist device 120 is not attached. In the present embodiment, when the walking state determination unit 210c determines that the leg to which the walking assist device 120 is attached has switched from the standing state to the swinging state, the bending-extension control unit 210d starts the bending control for the swinging state by the walking assist device 120.

Here, the response performance of the load distribution sensor 222 when the load distribution sensor 222 is loaded is higher than that when the load distribution sensor 222 is unloaded. Therefore, the walking state determination unit 210c can accurately detect the switching timing of the leg (affected leg) to which the walking assist device 120 is attached from the standing state to the swinging state. That is, the walking state determination unit 210c can accurately determine the walking state of the trainee 900. Further, the bending-extension control unit 210d can start the bending control for the swinging state by the walking assist device 120 at an appropriate timing. As a result, the trainee 900 can perform effective walking training.

### Example of Method of Determining Walking State of Trainee 900

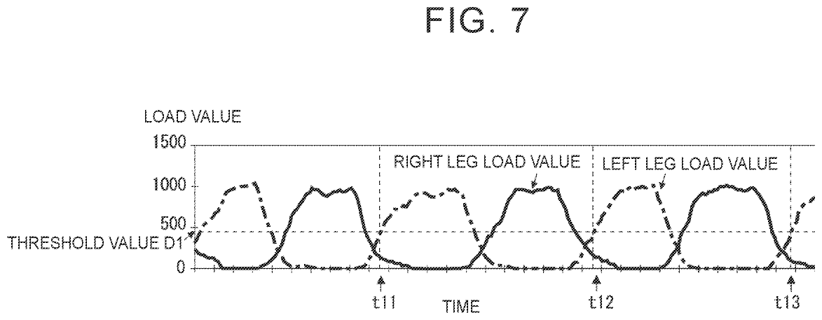

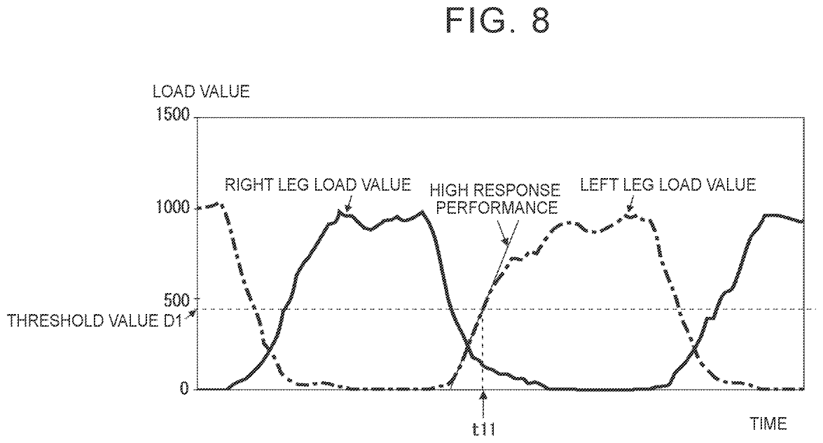

FIG. 7 is a timing chart showing an example of a method of determining the walking state of the trainee 900 by the walking training device 100. FIG. 8 is a timing chart obtained by enlarging a part of FIG. 7. In the example of FIGS. 7 and 8, a case where the right leg is the affected leg to which the walking assist device 120 is attached and the left leg is a healthy leg will be described. Further, in the example of FIGS. 7 and 8, the detection of the switching timing of the right leg, which is the affected leg, from the standing state to the swinging state will be mainly described.

Referring to FIG. 7, the walking state determination unit 210c determines that the right leg (affected leg) has switched from the standing state to the swinging state at the timing when the load value of the left leg (healthy leg) detected by the load distribution sensor 222 increases to a value equal to or more than a threshold value D1 (timing when the load value changes from the value less than the threshold value D1 to the value equal to or more than the threshold value D1) (time t11, t12, t13). When the walking state determination unit 210c determines that the right leg (affected leg) has switched from the standing state to the swinging state, the bending-extension control unit 210d starts the bending control for the swinging state by the walking assist device 120 attached to the right leg.

Here, as can be seen from the timing chart of FIG. 8 obtained by enlarging the vicinity of time t11, the response performance of the load distribution sensor 222 when the load distribution sensor 222 is loaded is higher than that when the load distribution sensor 222 is unloaded. Therefore, the walking state determination unit 210c can accurately detect the switching timing of the leg (affected leg) to which the walking assist device 120 is attached from the standing state to the swinging state. That is, the walking state determination unit 210c can accurately determine the walking state of the trainee 900. Further, the bending-extension control unit 210d can start the bending control for the swinging state by the walking assist device 120 at an appropriate timing. As a result, the trainee 900 can perform effective walking training.

The threshold value D1 can be set to any load value depending on the trainee 900. For example, the threshold value D1 is set to a predetermined load value through the operation of the trainee 900 or the training staff 901. Specifically, the threshold value D1 is set to a load value of, for example, about 5 kg. Alternatively, the threshold value D1 may be set to a load value at a predetermined ratio of the maximum value of the load received from the affected leg (load value received from the affected leg when standing only on the affected leg). Specifically, the threshold value D1 may be set to, for example, a load value of about 60% of the maximum value of the load received from the affected leg.

Alternatively, the threshold value D1 may be set to a load value at a predetermined ratio of the total value of the loads received from both legs. At this time, the walking state determination unit 210c determines that the right leg, which is the affected leg, has switched from the standing state to the swinging state, at the timing when the load received from the left leg, which is a healthy leg, changes from a value at less than the predetermined ratio of the total value of the loads received from both legs to a value at the predetermined ratio or more, for example.

### Another Example of Method of Determining Walking State of Trainee 900

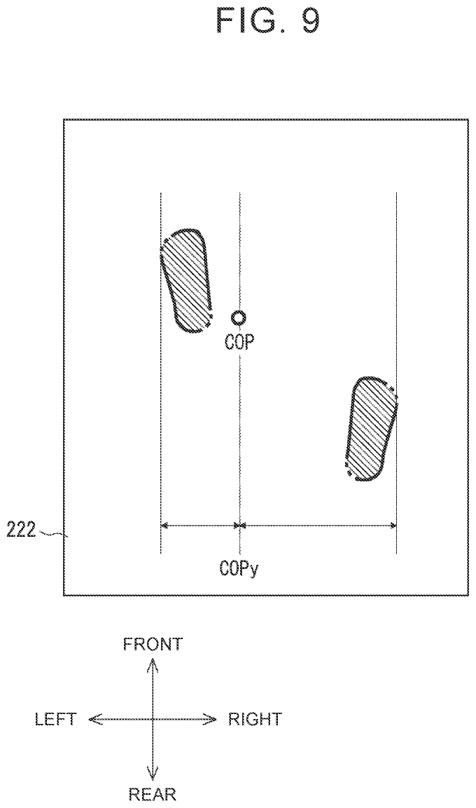

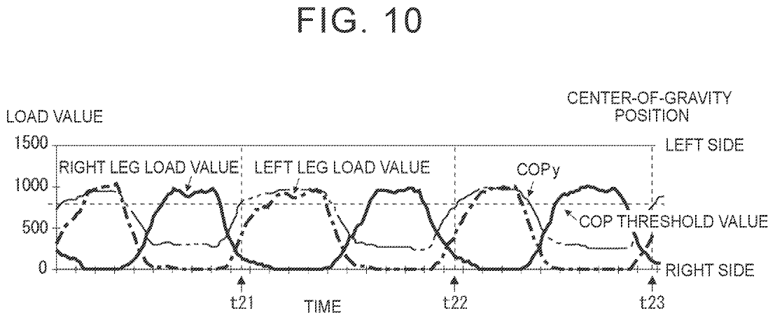

FIG. 9 is a schematic plan view illustrating another example of the method of determining the walking state of the trainee 900 by the walking training device 100. FIG. 10 is a timing chart showing the other example of the method of determining the walking state of the trainee 900 by the walking training device 100. In the example of FIGS. 9 and 10, a case where the right leg is the affected leg to which the walking assist device 120 is attached and the left leg is a healthy leg will be described. Further, in the example of FIGS. 9 and 10, the detection of the switching timing of the right leg, which is the affected leg, from the standing state to the swinging state will be mainly described.

As shown in FIG. 9, first, the walking state determination unit 210c identifies a center-of-gravity position COP of the trainee 900 from the load detected by the load distribution sensor 222. Here, the center-of-gravity position COP approaches the ground contact region of the left leg as the left leg load increases with respect to the right leg load, whereas the center-of-gravity position COP approaches the ground contact region of the right leg as the right leg load increases with respect to the left leg load. Therefore, it is possible to determine whether the right leg (affected leg) has switched from the standing state to the swinging state, based on the degree of approach of the center-of-gravity position COP toward the left leg (healthy leg) side (that is, the state of increase in the load received from the left leg).

For example, the degree of approach of the center-of-gravity position COP toward the left leg when the center-of-gravity position COP is located in the ground contact region of the right leg (that is, when standing only on the right leg) is set to 0%. The degree of approach of the center-of-gravity position COP toward the left leg when the center-of-gravity position COP is located in the ground contact region of the left leg (that is, when standing only on the left leg) is set to 100%. At this time, referring to FIG. 10, the walking state determination unit 210c determines that the right leg has switched from the standing state to the swinging state at the timing when the degree of approach of the center-of-gravity position COP toward the left leg reaches, for example, 80% (time t21, t22, t23). In the example of FIG. 10, a component COPy of the center-of-gravity position COP in the right-left direction is shown. When the walking state determination unit 210c determines that the right leg (affected leg) has switched from the standing state to the swinging state, the bending-extension control unit 210d starts the bending control for the swinging state by the walking assist device 120 attached to the right leg.

Even with such a determination method, the walking state of the trainee 900 can be accurately determined. Therefore, the bending control for the swinging state by the walking assist device 120 can be started at an appropriate timing. As a result, the trainee 900 can perform effective walking training.

As described above, the walking training device 100 according to the present embodiment detects that the leg (affected leg) to which the walking assist device 120 is attached has switched from the standing state to the swinging state based on the state of increase in the load received by the load distribution sensor 222 from the leg (healthy leg) to which the walking assist device 120 is not attached, and starts the bending control for the swinging state by the walking assist device 120. Here, the response performance of the load distribution sensor 222 when the load distribution sensor 222 is loaded is higher than that when the load distribution sensor 222 is unloaded. Therefore, the walking training device 100 can accurately detect the switching timing of the leg (affected leg) to which the walking assist device 120 is attached from the standing state to the swinging state. That is, the walking training device 100 can accurately determine the walking state of the trainee 900. Further, the walking training device 100 can start the bending control for the swinging state by the walking assist device 120 at an appropriate timing. As a result, the trainee 900 can perform effective walking training.

In the present embodiment, the case where the walking assist device 120 is attached to the right leg has been described as an example, but the present disclosure is not limited to this. For example, the walking assist device 120 may be attached to the left leg. In this case, the walking training device 100 detects that the left leg has switched from the standing state to the swinging state based on the increase in the load received by the load distribution sensor 222 from the right leg, and starts the bending control for the swinging state by the walking assist device 120 attached to the left leg. Alternatively, the walking assist device 120 may be attached to each of the right leg and the left leg. In this case, the walking training device 100 detects that the left leg has switched from the standing state to the swinging state based on the state of increase in the load received by the load distribution sensor 222 from the right leg and starts the bending control for the swinging state by the walking assist device 120 attached to the left leg, as well as detects that the right leg has switched from the standing state to the swinging state based on the increase in the load received by the load distribution sensor 222 from the left leg and starts the bending control for the swinging state by the walking assist device 120 attached to the right leg.

In the present embodiment, the case has been described in which the switching of one leg to which the walking assist device 120 is attached from the standing state to the swinging state is detected based on the state of increase in the load received by the load distribution sensor 222 from the other leg, but the present disclosure is not limited to this. As a matter of course, the switching of one leg to which the walking assist device 120 is not attached from the standing state to the swinging state can also be detected based on the state of increase in the load received by the load distribution sensor 222 from the other leg.

Further, in the present disclosure, part or all of the processes in the walking training device 100 can be realized by causing a central processing unit (CPU) to execute a computer program.

The above program includes instructions (or software codes) for causing the computer to perform one or more of the functions described in the embodiments when loaded into the computer. The program may be stored in a non-transitory computer-readable medium or a tangible storage medium. Examples of the non-transitory computer-readable medium or the tangible storage medium include, but are not limited to, a random-access memory (RAM), a read-only memory (ROM), a flash memory, a solid-stated drive (SSD) or other memory technologies, a compact disc read-only memory (CD-ROM), a digital versatile disc (DVD), Blu-ray (registered trademark) disc, or other optical disc storages, and a magnetic cassette, a magnetic tape, a magnetic disc storage or other magnetic storage devices. The program may be transmitted on a transitory computer-readable medium or a communication medium. Examples of the transitory computer-readable medium or the communication medium include, but are not limited to, electrical, optical, acoustic, or other forms of propagating signals.

What is claimed is:

1. A walking training system comprising:
a robot leg attached to one leg of a trainee;
a treadmill;
a load distribution sensor that is attached to the treadmill and detects a distribution of a load received from a sole of the trainee riding on a belt of the treadmill;
a walking state determination unit that determines whether the one leg has switched from a standing state to a swinging state based on a state of increase in a load detected by the load distribution sensor and received from another leg of the trainee performing walking training; and
a control unit that starts bending control for the swinging state of the one leg by the robot leg when the walking state determination unit determines that the one leg has switched from the standing state to the swinging state.

2. The walking training system according to claim 1, wherein the walking state determination unit determines that the one leg has switched from the standing state to the swinging state when the load received from the other leg becomes equal to or more than a predetermined load.

3. The walking training system according to claim 1, wherein the walking state determination unit determines that the one leg has switched from the standing state to the swinging state when the load received from the other leg becomes equal to or more than a predetermined ratio of a maximum value of a load received from the one leg.

4. The walking training system according to claim 1, wherein the walking state determination unit determines that the one leg has switched from the standing state to the swinging state when a center-of-gravity position of the load detected by the load distribution sensor comes in a predetermined region including a position of the other leg.

5. A method for controlling a walking training system, the method comprising:
a step of using a load distribution sensor attached to a treadmill to detect a distribution of a load received from a sole of a trainee riding on a belt of the treadmill;
a step of determining whether one leg to which a robot leg is attached has switched from a standing state to a swinging state based on a state of increase in a load received from another leg that is different from the one leg; and
a step of starting bending control for the swinging state of the one leg by the robot leg when determining that the one leg has switched from the standing state to the swinging state.

6. A control program that causes a computer to execute:
a process of using a load distribution sensor attached to a treadmill to detect a distribution of a load received from a sole of a trainee riding on a belt of the treadmill;
a process of determining whether one leg to which a robot leg is attached has switched from a standing state to a swinging state based on a state of increase in a load received from another leg that is different from the one leg; and
a process of starting bending control for the swinging state of the one leg by the robot leg when determining that the one leg has switched from the standing state to the swinging state.
